(19) United States
(12) Patent Application Publication
(10) Pub. No.: US 2005/0131371 A1
(43) Pub. Date: Jun. 16, 2005

(54) Fold line resistant absorbent articles

(76) Inventors: David A. Fell, Neenah, WI (US); Paige A. Dellerman, Appleton, WI (US); James R. Bath, Appleton, WI (US)

Correspondence Address: BRINKS HOFER GILSON & LIONE, P.O. BOX 10395, CHICAGO, IL, 60610, US

(21) Appl. No.: 10/732,913

(22) Filed: Dec. 10, 2003

(51) Int. Cl. A61F 13/15; A61F 13/20

(52) U.S. Cl. 604/385.02

(57) ABSTRACT

An absorbent system includes a packaging component and an absorbent article that is removably disposed in the packaging component. The absorbent article includes at least first and second portions, each defining a body side surface. The absorbent article is independently movable from a folded condition, wherein the body side surfaces of the first and second portions face each other in a substantially parallel relationship under an applied pressure applied to the first and second portions, to an unfolded condition, wherein no pressure is applied to the first and second portions. The body-side surfaces of the first and second portions form at least one angle greater than 90° in the unfolded condition when the absorbent article is positioned in the folded condition under 2800 psi of applied pressure for about 5 seconds and then is released to the unfolded condition for about 20 seconds. In yet another aspect, the absorbent article further includes a third portion having a body-side surface. The body-side surfaces of the first and third portions are folded to face the body-side surface of said second portion, producing a tri-folded article.

### BACKGROUND

The present invention relates generally to absorbent articles for absorbing and retaining human exudates, and in particular, to an absorbent article that is folded when packaged and which resists the permanent deformation generally associated with folded and wrapped absorbent articles.

Absorbent articles such as sanitary napkins, incontinent garments, incontinence shields, and the like are designed to be worn adjacent to a user's pudendum to absorb body fluids such as menses, blood, urine, and other excrements. Many women carry extra sanitary napkins with them in purses, glove compartments, or brief cases. Because an individual unprotected sanitary napkin may become soiled while being transported from place to place, many absorbent articles are individually packaged and are folded prior to being packaged.

When traditional absorbent materials are used, the folding process typically results in permanent creasing of the product and the absorbent core. The creasing can impair product performance in several ways including pad cracking, leakage, and consumer discomfort. In the case of trifolded pads and pantiliners, creasing may also produce a less finished appearance and make it more difficult for the consumer to place the product in her underwear.

Accordingly, the present invention is an absorbent article that will remedy this, and other, problems of the prior endeavors. These attributes will become clear as the present invention is more thoroughly discussed in this application.

### SUMMARY

Briefly stated, in one aspect, an absorbent system includes a packaging component and an absorbent article that is removably disposed in the packaging component. The absorbent article includes at least first and second portions, each defining a body side surface. The absorbent article is independently movable from a folded condition, wherein the body side surfaces of the first and second portions face each other in a substantially parallel relationship under an applied pressure applied to the first and second portions, to an unfolded condition, wherein no pressure is applied to the first and second portions. The body-side surfaces of the first and second portions form at least one angle greater than 90 degrees (90°) in the unfolded condition when the absorbent article is folded and enclosed in a pouch during the manufacturing process or alternatively is positioned in the folded condition under 19300 kilopascals (kPa) (2800 psi (pounds per square inch)) of applied pressure for 5 seconds at 21° (degrees) C (Celsius) and then is released to the unfolded condition for about 20 seconds.

In yet another aspect, the absorbent article further includes a third portion having a body-side surface. The body-side surfaces of the first and third portions may be folded to face the body-side surface of said second portion, producing a tri-folded article.

In yet another aspect, an absorbent article that resists permanent creasing includes an absorbent core. The absorbent core includes at least first and second portions that each defines a body side surface. The absorbent article is independently movable from a folded condition, wherein the body side surfaces of the first and second portions face each other in a substantially parallel relationship under an applied pressure applied to the first and second portions, to an unfolded condition, wherein no pressure is applied to the first and second portions. The body-side surfaces of the first and second portions of the present invention form at least one angle greater than 90° in the unfolded condition when the absorbent core is positioned in the folded condition under 19300 kPa (2800 psi) of applied pressure for about 5 seconds at 21° C. and then is released to the unfolded condition for about 20 seconds.

In another aspect, the absorbent core has a body-side absorbent layer and a garment side absorbent layer, wherein the body-side absorbent layer includes foam and the garment side absorbent layer includes a superabsorbent material.

In yet another aspect, a thin folded absorbent article, resistant to permanent creasing, includes an absorbent core that is independently movable between a folded and an unfolded condition. The absorbent core has an unfolded thickness of no more than 8 mm and includes a foam layer and a layer containing superabsorbent. The absorbent article has at least a first portion with a body-side surface and a second portion with a body-side surface. The body-side surfaces face one another when the absorbent article is in a folded condition. Also, when the absorbent core is unfolded, the body-side surfaces of the first and second body-side surfaces create at least one angle that is greater than 90°.

Yet another aspect includes a method of using an absorbent article which includes providing an absorbent article that is removably disposed in a packaging component in a folded condition. The absorbent article has at least first and second portions, each defining a body side surface. The absorbent article is independently movable from the folded condition in which the body side surfaces of the first and second portions face each other in a substantially parallel relationship under an applied pressure such as being enclosed in a pouch during the manufacturing process. In an unfolded condition, no pressure is applied to the first and second portions and the body-side surfaces of the first and second portions form at least one angle greater than 90°. The absorbent article is positioned in said folded condition under 19300 kPa (2800 psi) of said applied pressure for about 5 seconds at 21° C. and then is released to said unfolded condition for about 20 seconds. The article then grasped by the user, removed from the packaging component and allowed to independently move from the folded condition to the unfolded condition.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

FIG. 1 is a plan view of one embodiment of an absorbent article of the present invention in an unfolded configuration.

FIG. 2 is a plan view of another embodiment of an absorbent system of the present invention including a packaging component and an absorbent article in a folded configuration.

FIG. 3 is an exploded view of an embodiment of an absorbent article of the present invention.

FIG. 4 is a side view of an embodiment of an absorbent article of the present invention in a tri-folded configuration.

FIG. 5 is a side view of an embodiment of an absorbent article of the present invention in an unfolded configuration.

FIG. 6 is a side view of a Conventional article in an unfolded configuration.

FIG. 7 is a plan view of a Conventional Absorbent article in an unrolled configuration.

FIG. 8 is a plan view of an embodiment of an absorbent article of the present invention in an unrolled configuration.

### DETAILED DESCRIPTION OF THE INVENTION

As used herein, the term “airlaid” refers to the process of producing an absorbent material where unlike components are conveyed in an air-stream and homogenously mixed or provided in a stratified configuration and then bonded together. For example, this may include, but is not limited to, the mixture of pulp fibers, synthetic fibers, superabsorbent materials and binder material. The binder material is often, but not limited to, synthetic bicomponent binder fibers and or latexes. There are a number of commercial processes available to produce airlaid absorbent structures. For example, airlaid processes are available from Danweb Corp. having offices in Risskov, Denmark, and from M&J Forming Technologies having offices in Horsens, Denmark. Examples of suitable products and the process for forming them are described in U.S. Pat. No. 4,640,810 to Laursen et. al., U.S. Pat. No. 4,494,278 to Kroyer et. al., U.S. Pat. No. 4,351,793 to Day, and U.S. Pat. No. 4,264,289 to Day, the relevant portions of which are incorporated by reference.

An airlaid process provides a mixture of raw materials and the ability to add synthetic fibers and/or binder agents to the mixture to stabilize the resultant absorbent. As a stabilizer, binders reduce the amount of wet collapse in the structure and maintain a lower density in the saturated state. That is, the binder assists the absorbent matrix in maintaining its integrity even under load or while saturated. In addition, the resulting structure has both a higher dry and wet tensile strength than a corresponding structure without a binding agent.

The term “body-side” should not be interpreted to mean in contact with the body of the user, but rather simply means the side that would face toward the body of the user, regardless of whether an undergarment is actually being worn by the user and regardless of whether there are or may be intervening layers between the component and the body of the user. Likewise, the term “garment-side” should not be interpreted to mean in contact with the garments of the user, but rather simply means the side that faces away from the body of the user, and therefore toward any outer garments that may be worn by the user, regardless of whether the undergarment is actually being worn by a user, regardless of whether any such outer garments are actually worn and regardless of whether there may be intervening layers between the component and any outer garment.

As used herein, the “crotch region” of an absorbent article refers to the generally central region that will be in contact with the crotch of a user, near the lowermost part of the torso, and resides between the front and rear portions of the article. Typically, the crotch region generally spans approximately 7 to about 20 centimeters (cm) in the longitudinal direction depending on the function of the absorbent article.

It should be understood that the term “longitudinal,” as used herein, means of or relating to the length or the lengthwise direction. The term “laterally,” as used herein, means situated on, directed toward or running from side to side in a direction substantially perpendicular to the lengthwise direction.

The phrase “removably disposed” refers to one or more elements being placed within and surrounded by an outer element, such as a packaging component. The inner elements tend to remain within the outer element absent a separation force that may be applied to the outer element and where the outer element and the inner element are capable of being separated upon the application of a separation force. The required separation force is typically beyond that encountered through normal transportation of the absorbent system.

The term “superabsorbent” refers to a water-swellable, water-insoluble, organic or inorganic material capable, under the most favorable conditions, of absorbing at least about 15 times its weight and, more desirably, at least about 30 times its weight of an aqueous solution containing 0.9 weight percent sodium chloride. The super absorbent materials can be natural, synthetic, and modified natural polymers and materials. In addition, the super absorbent materials can be inorganic materials, such as silica gels, or organic compounds such as cross-linked polymers. Examples of superabsorbents are disclosed in U.S. Pat. No. 5,609,588 to DiPalma et. al., column 8, line 1 to line 57, which portion is incorporated herein by reference. A further example of a superabsorbent material is FAVOR SXM-880 available from Stockhausen Inc. 2401 Doyle Street, Greensboro, N.C. 27406, USA.

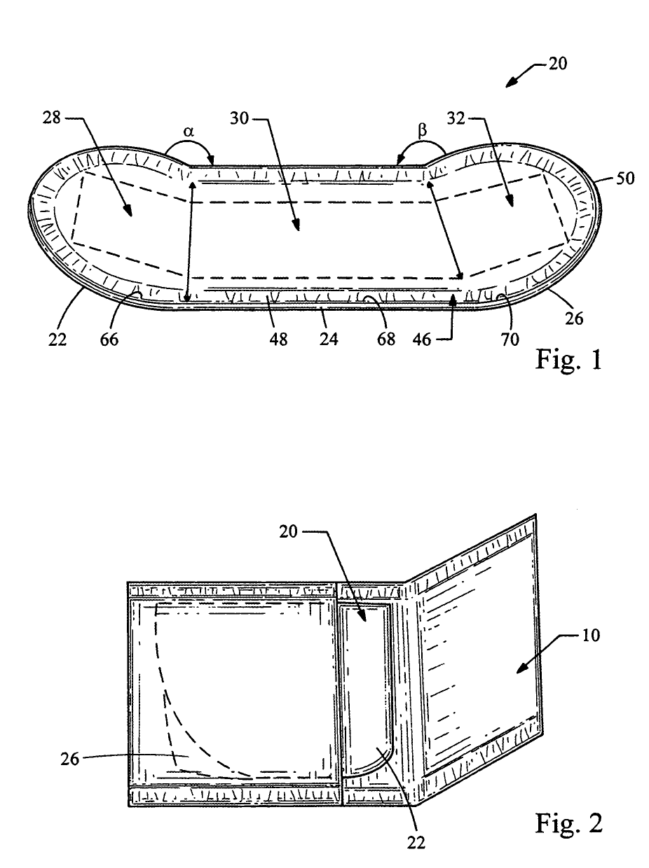

Referring to FIGS. 1 and 2, an absorbent system includes a packaging component 10, such as a pouch, and an absorbent article 20 that is resistant to creasing. The term “packaging component” refers to the portion of the system that encases the absorbent article 20 to protect it from being soiled before use. The packaging component is desirably a water impermeable material, such as a polypropylene film or nonwoven. An absorbent article 20 can be commercially folded and placed in a pouch using a folder, wrapper, and sealing unit such as one manufactured by the Curt G. Joa Company, Inc. of 100 Crocker Avenue, P.O Box 903, Sheboygan Falls, Wis., 53085 according to JOA drawing number 134R2042.

The absorbent article 20, as seen best in FIG. 1, is independently movable from a folded position to an unfolded position. The absorbent article 20 is generally folded into three portions, or tri-folded, when disposed within the packaging component. Desirably, the article 20 will have at least two portions, a first portion 22 and a second portion 24, but more desirably, will have a first portion 22, a second portion 24, and a third portion 26. For the purposes of this application, the absorbent article 20 will be described as having three portions, but it should be understood that the article 20 may be folded into more or less than three portions.

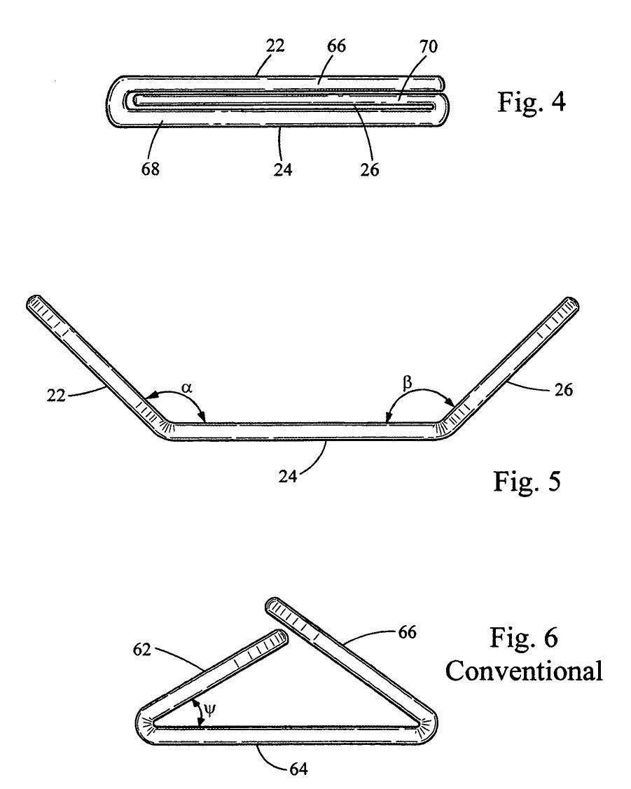

Generally speaking, when the absorbent article 20 is disposed within the packaging component 10 it may be in a folded condition. Each of the three portions has a body-side surface. Desirably, the article 20 is folded in a longitudinal direction. When folded, the body-side surfaces 28 and 32 of first portion 22 and third portion 26 face the body-side surface 30 of the second portion 24 in a substantially parallel relationship as shown in FIG. 4. The first and third portions 22 and 26 are independently movable from the folded condition to an unfolded condition. The term “independently movable” refers to the ability of the first and third portions 22 and 26 to move from the folded condition to an unfolded condition without the aid of any substantial pressure. The absorbent article 20 may be maintained in a folded condition by placing it in the packaging component 10, holding the article by hand or adhesive, or by applying outside pressure, such as from a press.

When outside pressure is removed from the absorbent article 20 of the invention, such as by removing the folded article from the pouch, the first and third portions 22 and 26 will independently move, in a longitudinal direction, from a folded condition to an unfolded condition, preferably without the user having to manually open up and smooth out the article before placing it in her underwear. When unfolded, the first 22 and second 24 and the second 24 and third portions 26 will create angles α and β of about between 90° and 180° degrees. See specifically FIG. 5. More desirably, the angles α and β will be about between 110° and 180°, and even more desirably, between 140° and 180°. The method of measuring the angles α and β will be discussed in more detail below.

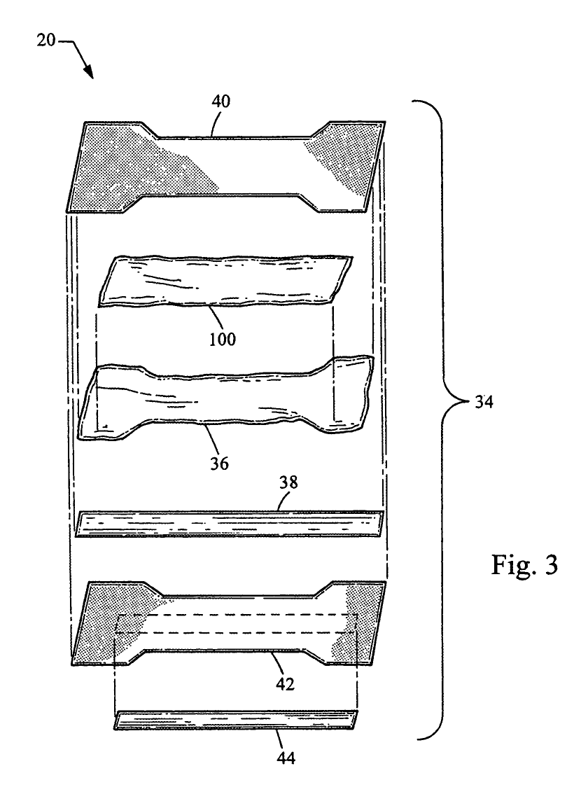

Referring now to FIGS. 1 and 3, the absorbent article 20 may include an absorbent core 34 with at least a body side absorbent layer 36 and an optional garment side absorbent layer 38. The absorbent core 34 may be positioned between a body side top sheet 40 and garment side baffle layer 42. The absorbent article 20 may also include an attachment means 44 that enables the user to removably attach the absorbent article 20 to the user's undergarments. The absorbent article may also include a transfer layer 100 between the topsheet 40 and the bodyside absorbent layer 36 (SEE FIG. 3).

Looking specifically at each of the article's 20 components, the top sheet 40 is designed to contact the body of the user and desirably is liquid-permeable. The top sheet 40 can be constructed of a woven or nonwoven material, from synthetic or natural materials and should be easily penetrated by body fluids. Suitable materials include spunbonded polyolefin nonwovens and bonded carded webs of polyester, polypropylene, polyethylene, nylon or other heat-bondable fibers. Other polyolefins, such as copolymers of polypropylene and polyethylene, linear low-density polyethylene, finely perforated film webs and net material also work well. A preferred cover material is a 17 grams per square meter (gsm) spunbonded liner. Spunbond webs are commercially available from Kimberly-Clark Corporation having an office at 401 N. Lake Street, Neenah, Wis., 54956. The top sheet 40 can also be constructed of a thermoplastic film that contains apertures and is flanked on both sides by a nonwoven material. This particular material provides a soft feel against a user's thigh while allowing body fluid to rapidly pass therethrough. Such materials are available from Tredegar Film Products with offices in Richmond, Va. 23225. The top sheet 40 can also be embossed to improve the aesthetic appearance of the absorbent article 20.

The garment side baffle layer 42 is generally liquid-impermeable and is designed to face the inner surface, i.e. the crotch portion of the user's underwear (not shown). The baffle 42 can be designed to permit a passage of air or vapor out of the absorbent article 20 while blocking the passage of liquids. The baffle 42 can be made from any material having the above identified properties. A suitable material is microembossed, polymeric film, such as polyethylene or polypropylene. One material that may be used is a polyethylene film having a thickness in the range of about 0.02 mm to about 1.0 mm and desirably about 0.03 mm to about 0.1 mm. Such materials are available from Pliant Corporation with offices located in Schaumberg, Ill.

The top sheet 40 and baffle 42 can be coextensive in a face-to-face contact around the outer edge of the absorbent core 34. The top sheet 40 and baffle 42 can be sealed together about their peripheries by use of an adhesive, by heat sealing, ultrasonics, or by any other process known in the art. A suitable hotmelt adhesive, used to seal the peripheries of the absorbent article 20, is Dispomelt 2525A and is commercially available from the National Starch Corp., Bridgewater, N.J. This sealed area is the peripheral seal 46. The peripheral seal 46 has end margins 48 and side margins 50. The length and width dimensions of the top sheet 40 and baffle layer 42 are generally larger than and extend beyond the corresponding dimensions of the absorbent core 34 to provide for the corresponding end margins 48 and side margins 50. The shape, length, and width, of the absorbent article 20 will be defined by the peripheral edges of the top sheet 40 and baffle layers 42.

The absorbent core 34 provides an absorbent structure that is configured for holding and storing absorbed liquids and other waste materials. The core 34 can be positioned and sandwiched between the top sheet 40 and the baffle layer 42 to form the absorbent article 20. Desirably, the absorbent core 34 is enclosed between the top sheet 40 and baffle layer 42 by the peripheral seal 46. The absorbent core 34 can be comprised of at least two discrete absorbent components: the body side absorbent 36 and the garment side absorbent 38. Alternatively, the absorbent core 34 can have only one component or can have more than two components.

The garment side absorbent layer 38 can be comprised of any material that will absorb bodily exudates such as menses, blood, and urine. Suitable absorbents include cellulose fluff, wood fluff, rayon, cotton, superabsorbents and mixtures thereof. Additionally stabilized absorbents such as airlaid can be used. Meltblown polymers, such as polyester, polypropylene, with staple cellulose fibers, can also be used. Desirably, a thin and flexible absorbent material, such as elastic coform, can be used. Elastic coform can be made according to the process disclosed in U.S. Pat. No. 6,362,389 to McDowall et. al, col. 11, lines 66-67 which refers to U.S. Pat. No. 4,818,464 to Lau and U.S. Pat. No. 4,100,324 to Anderson et. al. which are hereby incorporated by reference.

The body side absorbent layer 36 is desirably a resilient crease resistant material, such as open cell foam materials. Suitable foam materials are Rynel foam grade 562-R1 and Rynel grade foam 562B. Both foam materials are commercially available polyurethane open cell foams from Rynel Limited, Booth Bay, Me. The layers of the absorbent core may be, but are not necessarily, held together by a standard construction adhesive.

The body side 36 and the garment side 38 absorbent layers of the absorbent core 34 may have any suitable shape. Suitable shapes include a square, an hour-glass or bar bell shape, a rectangle, and a dogbone shape wherein the first portion 22 and third 26 portion are enlarged. The enlarged portions 22 and 26 are relatively flat with rounded profiles that are separated by a narrower center or second portion 24. The length and width dimensions of the absorbent layers 36 and 38 are less than the length and width dimensions of the top sheet 40 and baffle layer 42. The absorbent layers 36 and 38 have a thickness of about 0.1 millimeters (mm) to about 4.0 mm. Together, the total thickness of the top sheet 40, the baffle layer 42, and the absorbent layers 36 and 38, is about 2 mm to about 8 mm, desirably about 3 mm to about 5 mm. The bulk or thickness of the samples tested was tested at 1.40 kPa of pressure using a Digimatic Indicator Gauge, type DF 1050E, which is commercially available from Mitutoyo Corporation of Japan.

Referring now to FIGS. 4-6, FIG. 4 is a side view of an absorbent article 20 in a folded condition. Both conventional absorbent articles and absorbent articles 20 of the present invention may be folded in this manner. The first portion 22 may be folded so that its body side surface 28 is substantially parallel to the body side surface 30 of the second portion 24. Likewise, when an absorbent article 20 is tri-folded, the body-side surface 32 of the third portion 26 may be moved so that it is substantially parallel to the body side surface 30 of the second portion 24. The present invention should not be limited to this manner of folding.

When either a conventional tri-folded absorbent article or a crease resistant absorbent article, like those of the present invention, are allowed to unfold, each will have at least one angle α and if tri-folded, two angles α and β that are formed between the first 28 and second 30 bodyside surfaces and second 30 and third 32 body side surfaces of the portions 22, 24, and 26 of the absorbent article 20. For purposes of illustration, the angle between the first and second portion will be labeled α and the angle between the second and third portions will be labeled β.

As used in the present application, an obtuse angle is an angle that is greater than 90° and less than 180°. An acute angle is an angle that is less than 90°. As shown in FIG. 5, an article 20 of the present invention has at least a first and second portion 22 and 24 that forms a 90° or an obtuse angle α when they are allowed to unfold and stabilize. The conventional absorbent article 60, as shown in FIG. 6 has at least a first 62 and second portion 64 that forms an acute angle ψ when unfolded and allowed to stabilize. Various reproductions of conventional articles and embodiments of the present invention were crease tested and the results of those tests are shown below.

### TEST EXAMPLES

Table 1 below describes the construction of the sample absorbent articles that were used in the crease resistance tests. The products were made with the dimensions of the POISE® Pantiliner manufactured by Kimberly-Clark Corporation with offices at 401 N. Lake Street, Neenah, Wis. 54956. The products were 75 mm wide by 190 mm long, in a dogbone shape. The transfer layer 100 is centered longitudinally and laterally on the product and was 35 mm by 170 mm. The bodyside absorbent layer was 45 mm wide at its center, 60 mm wide at its greatest width end, and 170 mm long. The bodyside absorbent layer had an area of 83.6 square centimeters (cm²). The garment side absorbent is a centered rectangle and is 40 mm by 150 mm. The baffle layers for all of the products were comprised of 0.025 mm thick (1 mil) white polyethylene film available from Pliant Corporation. Type 2525A hot melt adhesive available from National Starch & Chemicals located in Bridgeport, N.J. was used between the top sheet and the spunbond transfer layer and between the transfer layer and the body side absorbent layer. No adhesive was used between the two absorbent layers. Adhesive was applied to the baffle layer to secure it to the garment side absorbent layer and to the top sheet.

TABLE 1

| | | | | Body-side | Garment-side |
|---|---|---|---|---|---|
| Sample | Thickness (mm) | Top Sheet | Transfer Layer | Absorbent | Absorbent |
| Commercially | 3.26 | 22 gsm | 40 gsm thermal | 200 gsm | Two Layers 200 gsm |
| Available | | Sawabond 4346 | bonded carded | Concert Airlaid, | Concert |
| Pantyliner | | white (Sandler | web from Shalag | 30% SAP, 0.15 g/cc | Airlaid, 30% |
| (Poise | | Gmbh, | Shamier, | | Dow 2035 SAP, |
| Pantiliner) | | Germany) | Kibbutz Shamir, | | 0.15 g/cc |
| | | | Israel | | |
| Comparative | 4.81 | 0.5 osy PRISM | Spunbond, 30 gsm | 200 gsm | Two Layers 200 gsm |
| Example A | | RibKnit | | Concert Airlaid, | Concert |
| | | spunbond | | 30% SAP, 0.15 g/cc | Airlaid, 30% |
| | | | | | Dow 2035 SAP, |
| | | | | | 0.15 g/cc |
| Comparative | 4.29 | 0.5 osy PRISM | Spunbond, 30 gsm | 100 gsm | Airfomed |
| Example B | | RibKnit | | Concert Airlaid, | 40% Dow 2035 |
| | | spunbond | | 20% Binder, no | 60% ND-416, |
| | | | | SAP, 0.07 g/cc | 500 gsm, |
| | | | | | 0.32 g/cc |
| Comparative | 3.63 | 0.5 osy PRISM | None | 100 gsm | Airformed |
| Example C | | RibKnit | | Concert Airlaid, | 40% Dow 2035 |
| | | spunbond | | 20% Binder, no | 60% ND-416, |
| | | | | SAP, 0.07 g/cc | 500 gsm, |
| | | | | | 0.32 g/cc |
| Comparative | 3.90 | 0.5 osy PRISM | spunbond 30 gsm | 100 gsm | Elastic Coform |
| Example D | | RibKnit | | Concert Airlaid, | at 400 gsm, 15% |
| | | spunbond | | 20% Binder, no | KRATON |
| | | | | SAP, 0.07 g/cc | G2755, 10% |
| | | | | | Sulfatate H-J. |
| | | | | | 75% SXM-9394 |
| Comparative | 3.92 | 0.5 osy PRISM | spunbond 30 gsm | Elastic Coform | Elastic Coform |
| Example E | | RibKnit | | at 400 gsm, 15% | at 400 gsm, 15% |
| | | spunbond | | KRATON | KRATON |
| | | | | G2755, 10% | G2755, 10% |
| | | | | Sulfatate H-J, | Sulfatate H-J, |
| | | | | 75% SP 1394 | 75% SXM-9394 |
| Embodiment A | 3.39 | 0.5 osy PRISM | None | Rynel Grade | Elastic Coform |
| | | RibKnit | | 562 B Foam, | at 400 gsm, 15% |
| | | spunbond | | 2 mm thick | KRATON |
| | | | | | G2755, 10% |
| | | | | | Sulfatate H-J, |
| | | | | | 75% SXM-9394 |
| Embodiment B | 4.38 (two | 0.5 osy PRISM | None | Rynel Grade | Elastic Coform |
| | measurements | RibKnit | | 562-R Foam, 3 mm | at 400 gsm, 15% |
| | taken) | spunbond | | thick | KRATON |
| | | | | | G2755, 10% |
| | | | | | Sulfatate H-J, |
| | | | | | 75% SXM-9394 |

In Table 1, all thickness measurements are an average of three measurements.

The top sheet of all tested embodiments was constructed of PRISM RibKnit which is available from the Kimberly-Clark Corporation with offices in Neenah, Wis. KRATON G2755 is a synthetic rubber blend commercially available from Kraton, Inc. of Houston, Tex. Sulfatate H-J is a chemically treated southern hardwood Kraft pulp supplied by Rayonier Inc. of Jessup, Ga. Dow Drytech® 2035M is a superabsorbent available from Dow Chemical Co., Midland Mich. (Comparative examples A, B, and C). ND-416 is a densification pulp commercially available from Weyerhaeuser Company, Tacoma, Wash. The airlaid materials are commercially available from Concert Industries, Gatineau, Quebec. The binder fiber in the Concert Airlaids is 6 mm, 2 denier type T255 biocomponent fiber from KoSa Corp., of Houston, Tex. SXM-9394 superabsorbent is commercially available from Stockhausen, Inc., Greenboro, N.C.

For Comparative example C, the garment side absorbent is a standard airformed fluff/superabsorbent mixture. It can be produced on a conventional online absorbent drum by homogeneously mixing high levels of superabsorbent and fluff pulp in a forming chamber as described in U.S. Pat. Appln. Pub. US 2002/0156441 A1 to Sawyer et. al., the relevant portions of which are incorporated herein by reference. The manufacture of this material is described in U.S. application Ser. No. 10/327,836 to Fell et al., paragraphs 92-97, which is assigned to Kimberly Clark, the relevant portions of which are hereby incorporated by reference.

Functionality

Saline Intake and Flowback Test

The saline intake and flowback test is used to measure the fluid intake time and flowback of adult incontinence pads. The fluid intake time is measured by using a timing device and visually estimating the length of time required to absorb three individual fluid insults. The fluid is 0.9% by weight sodium chloride dissolved in deionized water along with about 0.004 g/liter FD&C Blue #1 dye to make the liquid more visible. The test is typically done at room temperature (about 21° C.).

Layers of appropriately sized blotting paper such as James River Verigood 100 lb white available from Georgia Pacific Corporation with offices located in Neenah, Wis. are provided under the specimen (an incontinence pad) to collect any testing fluid that may flow over the side of the specimen. Apparatus for conducting this test include a four ounce capacity funnel part number 06122-20 available from Cole-Parmer Instrument Company (www.coleparmer.com) or equivalent. Additionally, a test board (a cylinder with a 25.4 mm inside diameter mounted on a plexiglass plate that fits on top of a mounting board and the test sample is mounted between the plate and the board) available from Kimberly-Clark Corporation, a stopwatch, and a pump, syringe, or beaker to pour the liquid into the cylinder are required.

For small samples the liquid was poured into the test board cylinder tube by hand. The sample is placed in the test board and secured (by pressing) on the board to insure a secure seal. A five milliliter insult was poured into the tube and the stopwatch started. One skilled in the art will understand that the insult size or volume is typically adjusted to be appropriate to the product being tested. For example, a five milliliter insult volume is appropriate for an incontinence pantiliner such as POISE pantiliners produced by Kimberly-Clark Corporation).

As soon as the fluid was totally absorbed (visual observation), the time was recorded. After one minute, the procedure was repeated for the second insult. After another minute, the procedure was repeated for a third 5 ml insult. A longer time means it takes that sample longer to absorb a fluid insult. Typically, lower times are better because the product tested will be less likely to leak in use.

After completing the third insult, the product is placed on a vacuum box (see equipment used in the Liquid Saturated Capacity Test, below) with the bodyside surface facing up. Appropriate amounts of pre-weighed blotter paper are placed over the product and it is then covered with a flexible rubber dam material. A vacuum of 3.5 kPa is drawn on the vacuum box for about 5 minutes. The pressure is then released and the blotter paper removed and weighed. The difference between the dry weight and the weight after the test is completed is the flowback.

Liquid Saturated Retention Capacity Test

The following test can be conducted to determine the amount of fluid retained by the absorbent core 16 and/or absorbent article 10. The liquid saturated retention capacity is determined as follows. The material to be tested, having a moisture content of less than about 7 percent by weight, is weighed and submerged in an excess quantity of a 0.9 weight percent aqueous saline solution at room temperature (about 21° C.). The material to be tested is allowed to remain submerged for 20 minutes. After the 20 minute submerging, the material is removed and placed on a TEFLON™ coated fiberglass screen having 0.25 inch (0.6 cm) openings (commercially available from Taconic Plastics Inc., Petersburg, N.Y.) which, in turn, is placed on a vacuum box and covered with a flexible rubber dam material. A vacuum of about 0.5 pound per square inch (about 3.5 kilopascals) is drawn on the vacuum box for a period of about 5 minutes with the use of, for example, a vacuum gauge and a vacuum pump). The material being tested is then removed from the screen and weighed.

The amount of liquid retained by the material being tested is determined by subtracting the dry weight of the material from the wet weight of the material (after application of the vacuum), and is reported as the absolute liquid saturated retention capacity in grams of liquid retained. If desired, the weight of liquid retained may be converted to liquid volume by using the density of the test liquid, and is reported as the liquid saturated retention capacity in milliliters of liquid retained. The lower the number, the less fluid the product can retain under pressure.

### Saline Intak and Flowback and Saturated Retention Capacity Tests

TABLE 2

| | Insult 1 | | | | Retention |
|---|---|---|---|---|---|
| | (sec- | Insult 2 | Insult 3 | Flowback | Capacity, |
| Sample | onds) | (seconds) | (seconds) | (g) | (g) |
| Poise | 9.6 | 27.68 | 37.45 | 4.23 | 45.3 |
| Pantiliner | | | | | |
| (Commercial) | | | | | |
| Serenity Dri- | 32.53 | 56.57 | 37.45 | 11.82 | 45.5 |
| Active | | | | | |
| (Commercial) | | | | | |
| Comparative | 8.18 | 14.26 | 16.26 | 6.02 | 53.1 |
| Example A | | | | | |
| Comparative | 9.73 | 10.43 | 15.56 | 2.59 | 57.6 |
| Example B | | | | | |
| Comparative | 21.3 | 15.18 | 22.7 | 4.21 | 57.7 |
| Example C | | | | | |
| Comparative | 9.07 | 17.67 | 20.47 | 3.04 | 60.7 |
| Example D | | | | | |
| Comparative | 14.65 | 34.26 | 39.72 | 13.2 | 112.5 |
| Example E | | | | | |
| Embodiment A | 31.33 | 46.07 | 52.5 | 5.24 | 59.7 |
| Embodiment B | 14.56 | 15.56 | 15.32 | 4.97 | 63.5 |

As shown in the above Table, the intake and absorbency properties of Embodiment A and B is substantially similar to the absorbency of conventional pantiliner products. Similar testing can be done for other incontinence products. For examples, POISE® Pads have capacities ranging from about 100 to about 400 grams. Other absorbent products such as diapers, child training pants and adult incontinence garments can have capacities ranging from about 300 grams up to 1500 grams and higher.

Intake rates can range from less than 1 ml/second up to 10 ml/second, or more depending on the desired use of the absorbent article.

Crease Testing

To test the crease resistancy of the embodiments of Table 1, the following procedures were used. Each sample was tri-folded by hand and placed under a hydraulic press, Carver Model 2518 S/N 2518-366, available from Carver, Inc. of Wabash, Ind. This press was used to simulate the fold lines of a machine made absorbent article. The samples were trifolded and pressed under conditions of 19,300 kPa (2800 psi) of pressure (actual pressure) with a pump speed of 100% at 21° C. The press was allowed to dwell for 5 seconds. Comparative Example A, which is similar to the commercial POISE pantiliner was folded, pressed under these conditions, and placed in a pouch for three days. When removed from the pouch and allowed to unfold, it resulted in angles similar to the commercial product (all acute). Based on this result, the Carver Press conditions delineated were used to fold and pouch all the handmade comparative examples and embodiments A and B. These conditions were selected because they successfully simulated the unfolding results obtained with commercially folded and pouched POISE pantiliners.

The absorbent articles were then removed from the pouch, opened, traced on paper and angles measured as described below. Each sample was removed from its pouch. Any areas where inadvertent adhesives or excess folds of backsheet material were holding the product together were carefully released without disturbing the trifolds of the pads. The force applied was just sufficient to eliminate sticking points, when necessary, and did not disturb the trifold of the entire article.

The articles were then placed, after about 20 seconds, on their longitudinal peripheral side and a trace of the planar body facing surfaces 66, 68, and 70 (See FIGS. 1 and 4) of first portion 22, second portion 24, and third portion 26, was made to show the angles formed between first portion 22, second portion 24, and third portion 26. The angle between the first portion 22 and the second portion 24 and the angles between the third portion 26 and the second portion 24 were measured using a protractor. Alternatively, the angle could be measured directly on the article. Two samples of the same construction were measured for each embodiment. The results of this measurement are recorded below in Table 3:

TABLE 3

| Sample | α° | β° |
|---|---|---|
| POISE Pantiliner 1 | 67 | 30 |
| POISE Pantiliner 2 | 76 | 35 |
| POISE Pantiliner 3 | 64 | 28 |
| Comparative Example A-1 | 65 | 55 |
| Comparative Example A-2 | 60 | 62 |
| Comparative Example B-1 | 62 | 67 |
| Comparative Example B-2 | 62 | 60 |
| Comparative Example C-1 | 73 | 68 |
| Comparative Example C-2 | 64 | 65 |
| Comparative Example D-1 | 62 | 54 |
| Comparative Example D-2 | 58 | 57 |
| Comparative Example E-1 | 62 | 55 |
| Comparative Example E-2 | 73 | 40 |
| Embodiment A-1 | 150 | 140 |
| Embodiment A-2 | 138 | 112 |
| Embodiment B-1 | 136 | 133 |
| Embodiment B-1 | 134 | 142 |

The results in Table 3 show that the conventional reproductions A-E remained folded, even after release from the folded condition and have angles α and β that are less than 90°. The angles of the conventional articles ranged from 28° to 76°. Embodiments A and B, however, create angles between their first portions 22 and second portions 24 and between their second portions 24 and third portions 26 that are greater than 90°. In fact, the angles are between 112° and 150°. The larger angles α and β of Embodiments A and B reduce, and in some instances, eliminate creasing of the folded article.

Rolled Testing

Another embodiment of the present invention includes a crease resistant rolled absorbent article. The absorbent articles listed in Table 1 were also subjected to crease resistancy testing by rolling the article, in the longitudinal direction. During manufacture, such absorbent articles may be rolled and then packaged in a pouch so that they may be transported or stored. The present invention would also eliminate creation of permanent deformation caused in the manufacture of rolled absorbent articles.

The samples were spirally rolled in the longitudinal direction by hand so that the first portion 22 and the third portion 26 of the absorbent article (See FIGS. 1 and 7) at least partially overlap one another other at least two times. An adhesive tape was used to secure the articles in the rolled position for three days. The tape was then cut and the article was placed on a flat surface to allow it to unroll on its own. The article was allowed to stabilize for 20 seconds.

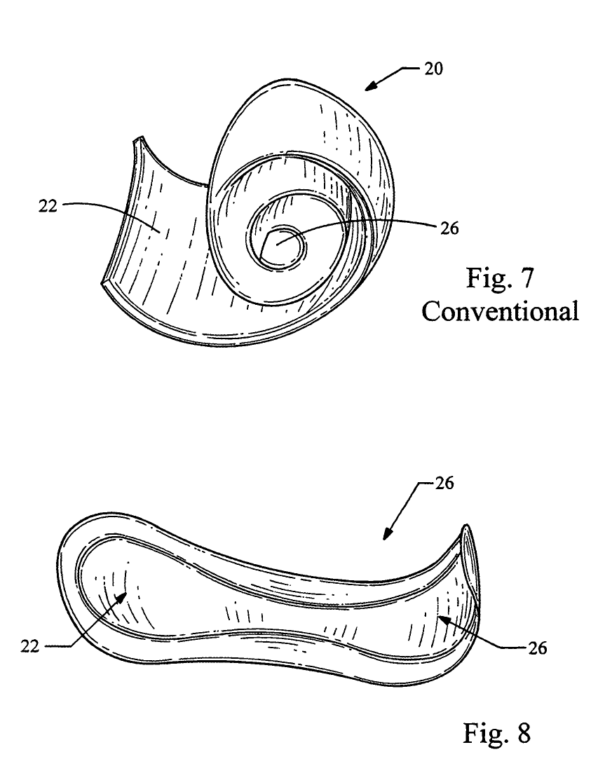

When allowed to unroll, the reproductions of the comparative examples and the conventional commercial articles remained in a spiral or rolled condition, as shown in FIG. 7. The first portion 22 and third portion 26 of these examples remained in at least a partially overlapped position when allowed to unroll and stabilize for 20 seconds. Embodiments A and B, however, nearly completely unrolled and the articles lay substantially parallel to the flat surface, as shown in FIG. 8, without overlap of their first portion 22 or third portions 26.

Another embodiment of the present invention includes a method of using the crease resistant absorbent article 20 and includes providing an absorbent article 20 that is removably disposed in a packaging component in a folded condition. The absorbent article 20 includes at least first portion 22 and second portion 24, each defining a body side surface 28 and 30, wherein the absorbent article 20 is independently movable from said folded condition.

In a folded condition the body side surfaces 28 and 30 of the first and second portions 22 and 24 face each other in a substantially parallel relationship when the portions are trifolded in a commercial folder/wrapper unit. When in a folded condition, the first and second portions 22 and 24 move to an unfolded condition, wherein no pressure is applied to the first and second portions 22 and 24, and body-side surfaces of the first and second portions 22 and 24 form at least one angle between about 90° to about 180°. More desirably, the angle is between 110° and 180° and more even desirably is approximately between about 140° and 180°. After the pressure is released, the portions are allowed to stabilize for about 20 seconds before being measured as previously described in the Crease Testing section.

To measure the angle α created by the first and second portions 22 and 24 and or second and third portions 24 and 26 of the article 20, the packaged article 20 is grasped by the user and is removed from the packaging component. The article 20 is allowed to independently move from the folded condition to an unfolded condition and then the angles are measured as previously described.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. An absorbent system comprising:
a packaging component; and
an absorbent article removably disposed in said packaging component, said absorbent article comprising at least first and second portions each defining a body side surface, wherein said absorbent article is independently movable from a folded condition, wherein said body side surfaces of said first and second portions face each other in a substantially parallel relationship under an applied pressure applied to said first and second portions, to an unfolded condition, wherein no pressure is applied to said first and second portions; and
wherein said body-side surfaces of said first and second portions form at least one angle greater than or equal to about 90° when said absorbent article is released to said unfolded condition for about 20 seconds.

2. The absorbent system of claim 1, wherein said body-side surfaces of said first and second portions form at least one angle greater than or equal to about 90° in said unfolded condition when said absorbent article is positioned in said folded condition under 2800 psi of said applied pressure for 5 seconds at 21° C. and then is released to said unfolded condition for about 20 seconds.

3. The absorbent system of claim 2, wherein said angle is greater than 110°.

4. The absorbent system of claim 2, wherein said angle is greater than 130°.

5. The absorbent system of claim 2, wherein the absorbent article comprises an absorbent core, the absorbent core having a body-side absorbent layer and a garment side absorbent layer, wherein the body-side absorbent layer comprises foam and the garment side absorbent layer comprises a superabsorbent material.

6. The absorbent system of claim 5, wherein the body-side absorbent layer of the absorbent article comprises an open celled foam.

7. The absorbent system of claim 2, wherein the absorbent article further comprises a third portion having a body-side surface, said body-side surfaces of said first and third portions being folded to face the body-side surface of said second portion.

8. The absorbent system of claim 2, wherein the absorbent article comprises an absorbent core, the absorbent core comprises a single absorbent layer.

9. The absorbent system of claim 2, wherein the absorbent article comprises an absorbent core, the absorbent core having at least two absorbent layers.

10. An absorbent article that resists permanent creasing comprising:
an absorbent core comprising at least first and second portions each defining a body side surface, wherein said absorbent article is independently movable from a folded condition, wherein said body side surfaces of said first and second portions face each other in a substantially parallel relationship under an applied pressure applied to said first and second portions, to an unfolded condition, wherein no pressure is applied to said first and second portions;
wherein said body-side surfaces of said first and second portions form at least one angle greater than or equal to about 90° in said unfolded condition when said absorbent core is positioned and maintained in said folded condition under 2800 psi of said applied pressure for about 5 seconds at 21° C. and then is released to said unfolded condition for about 20 seconds.

11. The absorbent article of claim 10, wherein said angle is greater than 110°.

12. The absorbent article of claim 10, wherein said angle is greater than 130°.

13. The absorbent article of claim 10, wherein said absorbent core further comprises a third portion having a body-side surface, said body-side surfaces of said first and third portions being folded to face the body-side surface of said second portion.

14. The absorbent article of claim 10, wherein said absorbent core comprises a body-side absorbent layer and a garment side absorbent layer, and wherein said body-side absorbent layer comprises foam and said garment side absorbent layer comprises a superabsorbent material.

15. The absorbent article of claim 14, wherein said body-side absorbent layer comprises an open celled foam.

16. A thin folded absorbent article resistant to permanent creasing comprising:
an absorbent core being independently movable between a folded and an unfolded condition and having a thickness of no more than 8 mm when in said unfolded condition, said absorbent core comprising:
a foam layer; and
a layer comprising superabsorbent;
wherein the absorbent core has at least a first portion with a body-side surface and a second portion with a body-side surface, said body-side surfaces facing one another when said absorbent article is in said folded condition, and
wherein when said absorbent core is moved from said folded condition to said unfolded condition, said body-side surfaces of said first and second portions form at least one angle greater than 90°.

17. The thin folded absorbent article of claim 16, wherein the absorbent core, when unfolded, has a thickness of less than 5 mm.

18. The thin folded absorbent article of claim 16, wherein the absorbent core, when unfolded after about 20 seconds, has a thickness of less than 4 mm.

19. The thin folded absorbent article of claim 16, wherein the angle is greater than 110°.

20. The thin folded absorbent article of claim 16, wherein the angle is greater than 130°.

21. The thin folded absorbent article of claim 16, wherein the absorbent article further includes a third portion having a body-side surface, said body-side surfaces of said first and third portions being folded to face the body-side surface of said second portion.

22. A thin folded absorbent article resistant to permanent creasing comprising:
an absorbent core being independently movable between a folded and an unfolded condition and having a thickness of no more than 8 mm when in said unfolded condition, said absorbent core comprising:
a first absorbent layer; and
a second absorbent layer;
wherein the absorbent core has at least a first portion with a body-side surface and a second portion with a body-side surface, said body-side surfaces facing one another when said absorbent article is in said folded condition, and
wherein when said absorbent core is moved from said folded condition to said unfolded condition, said body-side surfaces of said first and second portions form at least one angle greater than 90° when said absorbent article is positioned in said folded condition under 2800 psi of said applied pressure for 5 seconds at 21° C. and then is released to said unfolded condition for about 20 seconds.

23. The thin folded absorbent article of claim 22, wherein the first absorbent layer comprises and open celled foam.

24. The thin folded absorbent article of claim 22, wherein the second absorbent layer is comprised of superabsorbent.

25. A method of using an absorbent article comprising:
providing an absorbent article removably disposed in a packaging component in a folded condition, said absorbent article comprising at least first and second portions each defining a body side surface, wherein said absorbent article is independently movable from said folded condition, wherein said body side surfaces of said first and second portions face each other in a substantially parallel relationship under an applied pressure applied to said first and second portions, to an unfolded condition, wherein no pressure is applied to said first and second portions, wherein said body-side surfaces of said first and second portions form at least one angle greater than 90° in said unfolded condition when said absorbent article is positioned in said folded condition under 2800 psi of said applied pressure for about 5 seconds at 21° C. and then is released to said unfolded condition by;
grasping said absorbent article;
removing said absorbent article from said packaging component; and
allowing said absorbent article to independently move from said folded condition to said unfolded condition for about 20 seconds.

26. The method of claim 25, wherein said at least one angle is greater than 110°.

27. The method of claim 25, wherein said at least one angle is greater than 130°.

28. The method of claim 25, wherein the absorbent article comprises an absorbent core, the absorbent core having at least a body-side absorbent layer and a garment side absorbent layer, wherein the body-side absorbent layer comprises foam and the garment side absorbent layer comprises a superabsorbent material.

29. The method of claim 28, wherein the body-side absorbent layer of the absorbent article comprises an open celled foam.

30. The method of claim 25, wherein the absorbent article further comprises a third portion having a body-side surface, said body-side surfaces of said first and third portions being folded to face the body-side surface of said second portion.

31. The method of claim 25, wherein the absorbent article, when unfolded, has a thickness of less than 8 mm.

32. The method of claim 25, wherein the absorbent article, when unfolded, has a thickness of less than 5 mm.

33. An absorbent system comprising:
a packaging component; and
an absorbent article removably disposed in said packaging component, said absorbent article comprising at least first and second portions each defining a body side surface, wherein said absorbent article is independently movable from a rolled condition, wherein said body side surfaces of said first and third portions at least partially overlap one another at least two times under an applied pressure applied to said first and third portions, to an unrolled condition, wherein no pressure is applied to said first and third portions for approximately 20 seconds; and wherein said body-side surfaces of said first and third portions no longer overlap.

34. The absorbent article of claim 33, wherein said absorbent core comprises a body-side absorbent layer and a garment side absorbent layer, and wherein said body-side absorbent layer comprises foam and said garment side absorbent layer comprises a superabsorbent material.

35. The absorbent article of claim 33, wherein said body-side absorbent layer comprises an open celled foam.
